(19) United States
(12) Patent Application Publication
(10) Pub. No.: US 2024/0189143 A1
(43) Pub. Date: Jun. 13, 2024

(54) Hydrodynamically Actuated Preservative Free Dispensing System

(71) Applicant: Bausch + Lomb Ireland Limited, Dublin (IE)

(72) Inventor: Yehuda Ivri, Newport Coast, CA (US)

(73) Assignee: Bausch + Lomb Ireland Limited, Dublin (IE)

(21) Appl. No.: 18/583,268

(22) Filed: Feb. 21, 2024

Related U.S. Application Data
(63) Continuation of application No. 17/509,383, filed on Oct. 25, 2021, now Pat. No. 11,938,057.
Application No. 17/509,383 is a continuation-in-part of application No. 17/233,105, filed on Apr. 16, 2021, now Pat. No. 11,925,577.
(60) Provisional application No. 63/011,808, filed on Apr. 17, 2020.

(51) Int. Cl. A61F 9/00; A61B 17/00

(52) U.S. Cl. CPC A61F 9/0008; A61B 2017/00057; A61B 2017/00889

(57) ABSTRACT

Multi-dose preservative-free ocular fluid delivery devices are provided. The fluid delivery device includes a fluid dispensing system and a fluid package for storing a liquid therein and supplying said liquid to the dispensing system. The dispensing system comprises an elongated chamber which includes a check valve which defines a frontal closure to the chamber. The valve is normally closed and hermetically seals the chamber. The dispenser includes a vibration motor that induces oscillations to the chamber and to the fluid within. The oscillations of the chamber impart momentum to the fluid stored in the chamber which in turn imparts force that cyclically opens the valve to dispense streams or liquid droplets. Fluid is dispensed only when the motor oscillates while otherwise the valve is hermetically closed.

### CROSS REFERENCE TO RELATED APPLICATIONS

This application is a continuation patent application of U.S. patent application Ser. No. 17/509,383, filed Oct. 25, 2021, which is a continuation in part of U.S. patent application Ser. No. 17/233,105, filed Apr. 16, 2021, which claims the benefit of and priority to U.S. Provisional Patent Application No. 63/011,808, filed Apr. 17, 2020, each of which are hereby incorporated by reference in their entireties.

### FIELD OF THE INVENTION

The present invention generally pertains to devices for dispensing fluid medicines and more particularly pertains to such devices that store and deliver ophthalmic preservative-free medicines, specifically configured to increase the ease of use and enhance patient compliance with dosing instructions for the medicine.

### BACKGROUND

Ease of dispensing fluid medicines and compliance with dosing instructions are primary concerns with all patients. In particular, preservative-free dispensing bottles such as ophthalmic squeeze dispensers typically require greater actuation force due to a valve mechanism that seals the dispensing nozzle to prevent bacterial ingress and contamination. Such system requires much higher pressure to operate and hence much higher squeeze force is required. In addition, prior art dispensing bottles dispense only in an upside-down orientation which require an inconvenient head maneuver and which together with higher actuation force further increases the inconvenience.

Dispensers of the kind in question are known from the prior art, for example from U.S. Pat. Nos. 6,095,376, 9,676,525, US 2014/0336596, US 2016/0107180, U.S. Pat. Nos. 9,238,532, 8,056,766, 8,863,998, and 10,105,720. The dispenser shown in US 2014/0336596 comprises an outlet channel which connects the liquid reservoir to the outlet opening through an outlet valve which is arranged in the outlet channel and which opens when the bottle is squeezed and pressure is generated. Such preservative-free squeeze bottles typically require about 25-28N of squeeze force (Ophthalmic Squeeze Dispenser—Drug Development and Delivery October 2017 Vol. 17 No. 7 page 40). Elderly patients, or other patients lacking enough strength and/or dexterity in their hands, often experience problems dispensing medicine from such bottles.

This work provides preservative free ocular dispensing device that can be held horizontally, or in any convenient orientation while the actuation is done effortlessly by an electrical switch. This provides a cost effective solution that is consistent with standard drug packaging processes.

### SUMMARY

Multi-dose preservative-free ocular fluid delivery devices are provided. The fluid delivery device includes a fluid dispensing system and a fluid package for storing a liquid therein and supplying said liquid to the dispensing system. The dispensing system comprises an elongated chamber which includes a check valve which defines a frontal closure to the chamber. The valve is normally closed and hermetically seals the chamber. In this work the chamber includes a vibration motor that induces oscillations to the chamber and to the fluid within. The oscillations of the chamber impart momentum to the fluid stored in the chamber which in turn imparts force that cyclically opens the valve to dispense streams or liquid droplets. Fluid is dispensed only when the motor oscillates while otherwise the valve is hermetically closed.

The check valve can include a flexible plate which includes a conical aperture that extends through its thickness, the valve can further include a stationary spherical member that engages tangentially with the inner wall of the conical aperture to create a hermetic sealed closure. The plate can be made of elastomer that has a modulus of elasticity ranging between 0.1-1.2 GPa. The circumference of the plate can be attached to the chamber by a retaining ring that engages with the chamber in an interference fit to create the hermetically sealed closure.

The conical aperture extends through the thickness of the plate such that droplets are dispensed through the smaller opening of the aperture while the larger side of the aperture is in fluid communication with the chamber.

The spherical member may include an antibacterial coating which covers the area of the spherical member that is between the tangential engagement line and the small opening of the aperture.

The vibrational motor oscillates the chamber and the fluid within the chamber. Consequently, cycles of hydrodynamic pulses are generated causing the valve to cyclically open and dispense fluid. Here this phenomenon is characterized by oscillatory interactions between the valve and the surrounding fluid. The hydrodynamic force generated by the momentum of the fluid opens the valve and allows fluid flow through the aperture.

Fluid is dispensed only when the hydrodynamic force is sufficiently high to deform the aperture while otherwise the aperture hermetically seals the chamber. The system prevents ingress of microorganism into the chamber allowing storage of preservative free pharmaceutical. This work provides an electrically operated preservative-free dispensing system that is convenient and cost effective.

### BRIEF DESCRIPTION OF THE DRAWINGS

FIG. 1A is a cross sectional view of an embodiment of the invention.

FIG. 1B is a detail cross sectional view of the embodiment of FIG. 1A.

FIG. 1C is a 3D view of the embodiment of FIGS. 1A-B.

FIG. 2A is a side view and partial cross section view of an embodiment of the invention in operation.

FIG. 2B is a detail cross section view of the example of FIG. 2A.

FIG. 3 illustrates a perspective view of a vibration motor as used in embodiments of the invention.

FIG. 4A is a frontal view of an embodiment of the invention.

FIG. 4B is a side view of the embodiment of FIG. 4A.

FIG. 4C is a detail cross sectional view of the embodiment of FIG. 4A.

FIG. 5A is a frontal view of an exemplary device enclosed in a housing.

FIG. 5B is a side view of the example of FIG. 5A.

FIGS. 6A-B are perspective views of a device having a housing that includes a swivel cover.

FIGS. 7A-B show operation of an embodiment of the invention including an optical sensor for motor speed.

### DETAILED DESCRIPTION

This work describes dispensing devices and methods for delivery of preservative-free solutions or suspensions for ocular administration of ophthalmic drugs. The dispensing devices include a droplet ejecting system that is fluidly connected to an ampoule package containing a liquid to be dispensed. The droplet ejecting system includes a chamber having a check valve that defines a front closure to the chamber. The dispensing system further includes a vibration motor that oscillates the chamber and induces hydrodynamic pulses which consequently causes the valve to cyclically open and eject fluid droplets. The valve is normally closed and hermetically sealing the chamber. The valve opens exclusively in response to hydrodynamic pulses induced by the oscillation of the chamber. In this way fluid is dispensed only when the device is actuated while otherwise the aperture hermetically seals the device and prevents ingress of bacterial and microorganisms thereby allowing storage of preservative-free pharmaceutical formulation. The use of a vibration motor further enables convenient and cost effective, electronically controlled administration.

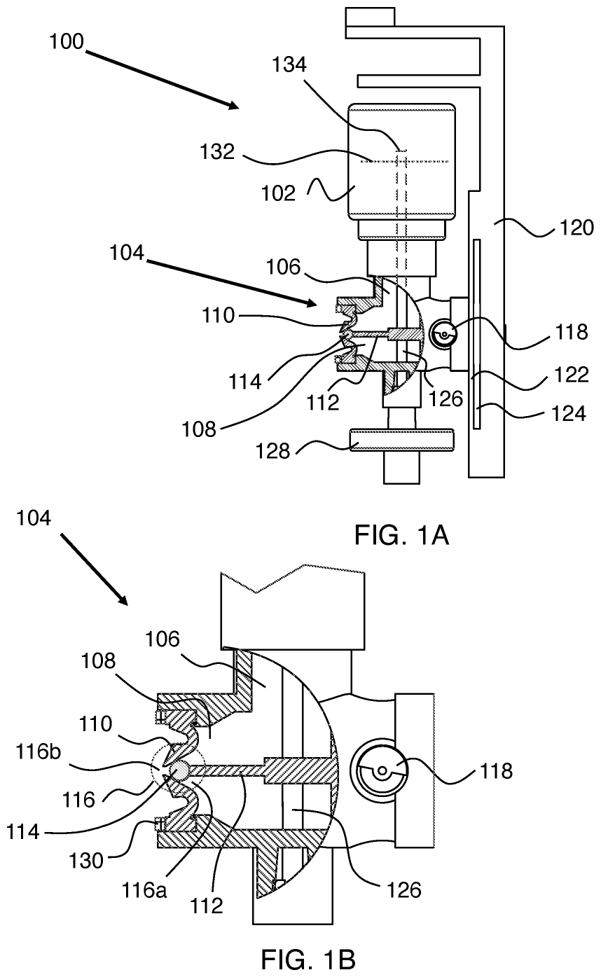

FIG. 1A and FIG. 1B illustrate a side view and an enlarged partial section view, respectively, of a fluid delivery device 100. Delivery device 100 includes a fluid reservoir 102 and dispensing system 104 connected to each other in fluid transmission relationship though passage 106. Dispensing system 104 includes a fluid chamber 108 and also includes a check valve including aperture plate 110 which provides frontal closure to the chamber 108. Referring to FIG. 1B it can be seen that aperture plate 110 includes a conical or tapered aperture 116 (shown inside a dotted circle for clarity) that extends through its center thickness and has a large inlet opening 116a in fluid communication with chamber 108 and a smaller exit opening 116b though which fluid droplets will be dispensed. Aperture plate 110 can be made of a flexible elastomer such as silicone rubber, VersaFlex, manufactured by VersaFlex Incorporated Kansas City, Kansas USA. Other elastomers with Young modulus of elasticity value between 0.5 GPa to 2 GPa can also be used. Dispensing system 104 further includes a stationary spherical member 114 that tangentially engages in pressure transmission relationship with the inlet opening 116a of the conical aperture providing hermetically sealed closure. In a preferred embodiment spherical member 114 is made of high density polyethylene (HDPE) that is harder than silicone thereby creating a tight closure as it engages with the softer aperture plate 110. Spherical member 114 preferably engages in pressure transmission relationship with the conical aperture 116a with a preload force of between 0.01N and 0.05N. The combination of aperture plate 110 and spherical member 114 provides a check valve as described above. Spherical member 114 is supported by pin member 112. Member 114 can have a shape other than spherical, since any shape capable of forming a good seal with aperture plate 110 can be used.

Aperture plate 110 can be retained to dispensing system 104 by a retaining ring 130, thereby creating a hermetically sealed closure.

Dispensing system 100 includes a venting tube 126 that is configured to equalize the pressure inside container 102 as the fluid is dispensed from the device. The opening 134 of venting tube 126 is extended above the fluid level 132 at any orientation that the device is held. Vent tube 126 can be connected via a 0.22 micron filter 128 to assure that the air that enters the device is sterile.

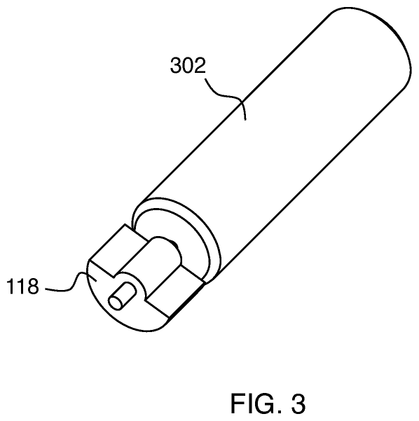

Dispensing system 100 further includes a vibration motor configured to oscillate chamber 108 and the fluid within the chamber. Here this motor is schematically shown as eccentric mechanical load 118 which vibrates the assembly as described when it is rotated by the motor (motor not shown in FIGS. 1A-B, but described below in connection with FIG. 3).

FIG. 1C is a 3D view of the embodiment of FIGS. 1A-B.

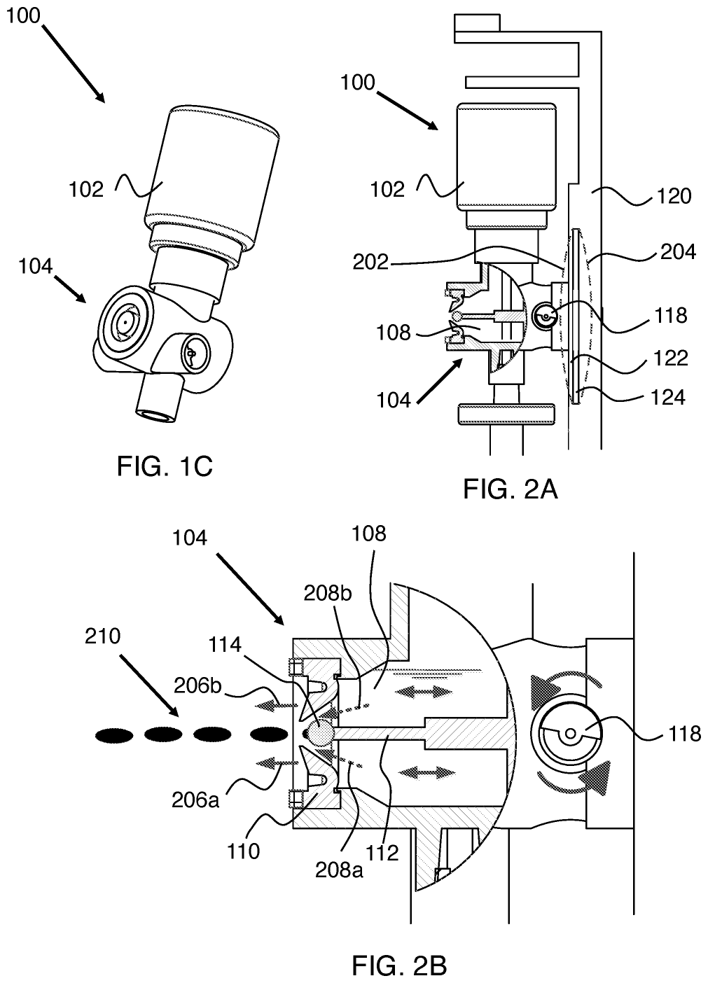

FIGS. 2A-B illustrate the response of the dispensing system 104 to oscillations. Vibrations of the motor induce oscillation to the body of dispensing system 104 which in turn generates fluid momentum within the chamber due to the dynamical interaction of the fluid with the solid structure of chamber 108. This phenomenon is often referred to as Solid-Fluid-Interaction (SFI). The momentum of the moving fluid exerts force that flexes the aperture plate 110 outwardly in the direction indicated by arrows 206a and 206b causing the aperture plate 110 to disengage from spherical member 114 thereby opening a fluid flow passage as indicated by the arrows 208a and 208b and ejection of fluid droplets 210.

Dispensing device 100 is supported by a flexible beam 122 or other structural embodiments which allows it to oscillate freely, as schematically shown by motion excursions 202 and 204. Preferably the spring constant of the beam 122 is 0.05 N/mm to 0.5 N/mm. For example, beam 122 can be formed by fabricating a slot 124 in support structure 120 such that the resulting beam 122 has a thickness suitable for providing a spring constant as recited above.

FIG. 3 illustrates an exemplary vibrational motor 302. The DC Motor 302 of this example has a cylindrical body with a diameter of 4 mm and total length of 17 mm. An eccentric flywheel 118 is attached to the motor shaft. The flywheel has a mass of 1.7 grams with a center of mass about 0.7 mm from the center of rotation. The motor receives 4.5-12 VDC and rotates at 6000-12000 RPM generating centrifugal force of 0.3N at a rotation speed of 8000 RPM. Other DC motors that generate centrifugal force of 0.1-1N and rotation speed of 1000-50000 RPM can be used. The motor can be controlled by a timer circuit which is set to provide an ON time as required to deliver a dose of 8-12 micro-liter. The actuation ON time is 60-200 ms depending on the rheology of the fluid in use. A timer circuit which incorporates a 555 timer IC or a microprocessor-based timer with a 12 volt battery such as A23 alkaline battery may be used.

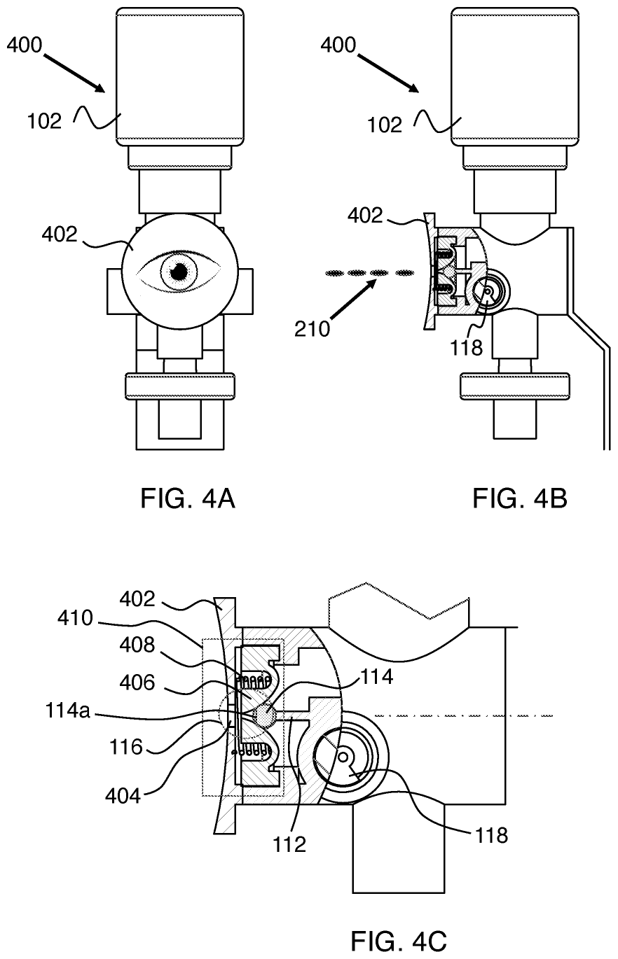

FIGS. 4A-C illustrate an alternative preferred embodiment of dispensing system 400. FIG. 4A illustrates a frontal view and FIG. 4B illustrates a side view of dispensing system 400. Dispensing system 400 includes a concave mirror 402 which assists in aligning device 400 and fluid stream 210 to the eye of the user. In use, dispensing device 400 is positioned in front of the eye such that the image of the eye appears sharply and in the center of mirror to the user. At this point the device is properly positioned in term of the distance of the eye from the nozzle 404 and its angular orientation relative to the eye. Upon actuation, stream 210 will be deposited precisely on the corneal surface of the eye. Device 400 preferably includes a 0.22 micron air filter configured to filter the vented air that flows into device 400, as in the previous example.

Device 400 includes a check valve having an aperture plate 406 with a conical aperture 116 that extends through its thickness. The check valve further includes a spherical member 114 that tangentially engages with the inlet opening of the conical aperture 116. In this example, the check valve also includes a compression spring 408 configured to force aperture plate 406 against spherical member 114. In this way a tight seal is created along the engagement line 114a, thus creating a tight and hermetic closure.

Spherical member 114 can be partially covered with an antimicrobial coating, specifically in the area of spherical member 114 that is not in contact with fluid in the chamber. The coated area thus extends between the engagement line 114a and the outlet of the conical aperture (i.e., to the left of 114a on FIG. 4C)). Examples of antimicrobial coatings include silver in metallic form, silver alloy or a non-metallic material that contains silver including salts of silver chloride and silver sulfadiazine. Other optional material includes biguanide derivatives, chlorohexidine diacetate and chlorohexidine digluconate.

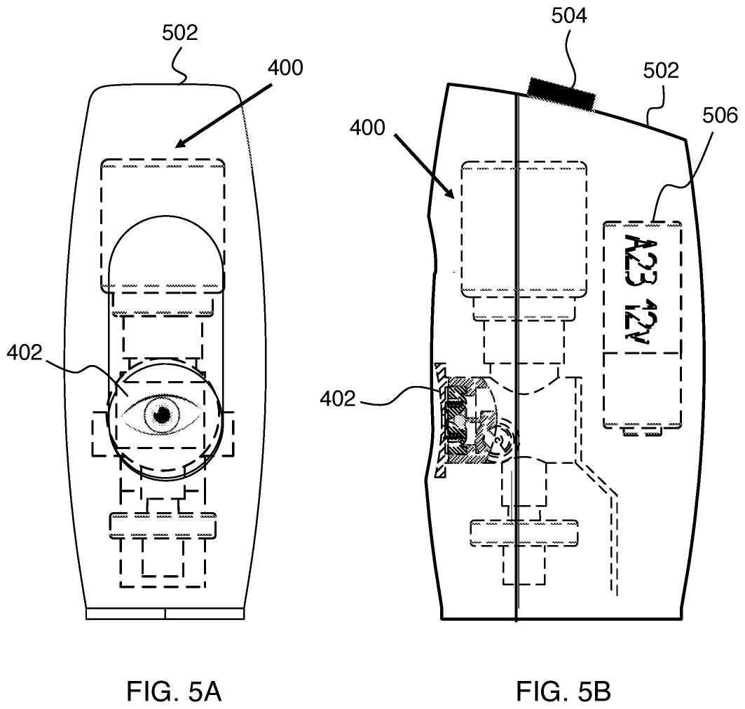

FIGS. 5A-B illustrate frontal and side views, respectively, of a dispensing device 400 packaged in a housing 502. Housing 502 provides a convenient enclosure to the dispensing system 400 that was described in relation in FIGS. 4A-C and is shown with dashed lines here. Housing 502 includes an electrical circuit (not shown) and a 12 volt battery (e.g., type A23) 506. The circuit controls the dispensing period such that a dose of 8-12 microliter is delivered to the ocular surface of the eye. Momentary switch 504 can be used to activate the device 400 such that a stream of droplets is ejected from the aperture as described earlier. FIGS. 5A-B also illustrate the mirror 402 which is visible on the front side of the device. Such a housing can also be used for a dispensing device 100 as described above.

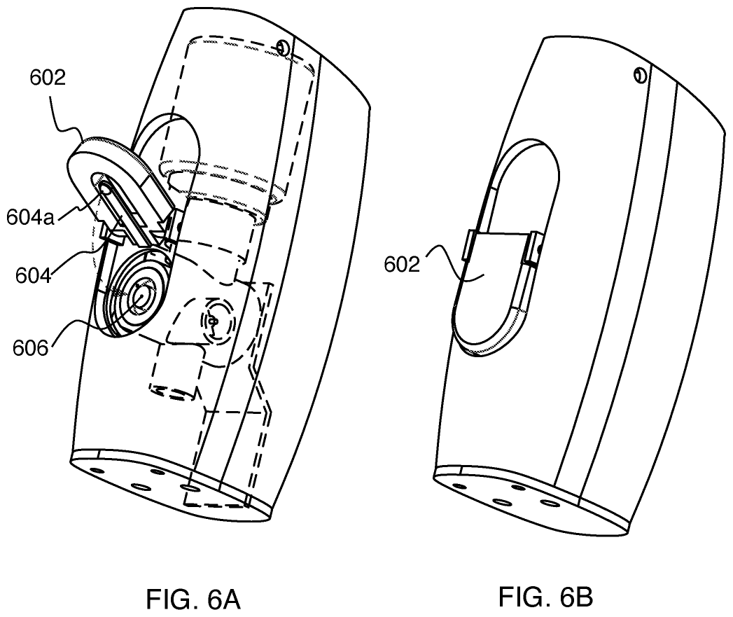

FIGS. 6A-B illustrate some preferred features of housings that can be used with embodiments such as device 100 and device 400 as described above. In this example, the housing includes a swivel cover 602 that covers dispensing nozzle 606 during periods of non-use. Swivel cover 602 provides a means to prevent bacterial contamination on the external areas where a residual fluid may be left following each use. Such residual fluid may contaminate subsequent stream as it is dispensed through the nozzle. Swivel cover 602 preferably includes a flexible member 604 that includes a surface 604a that is covered with antimicrobial coating. Surface 604a engages with the outlet opening of nozzle 606 when the swivel cover 602 is closed as illustrated in FIG. 6B. In this way antipathogen action is achieved. Alternatively, organic dyes with antiseptic action may also be used. For example, toluidine blue, methylene blue, gentian violet and acridine and related active substances such as acridine orange and acridine yellow as well as ethacridine lactate. Germicidal polymers such as polyhexanide are also possible. Materials that contain additives which contain metal-organic substances with an ionizing effect can also be used. Such additives are available from SteriOne GmbH Berlin, Germany. Examples of antimicrobial coatings that can be used on surface 604a include silver in metallic form, silver alloy or a non-metallic material that contains silver including salt of silver chloride and silver sulfadiazine. Other optional material includes biguanide derivatives, chlorohexidine diacetate and chlorohexidine digluconate.

### Closed Loop Motor Control

Optionally, the device includes a drive circuit that controls the rotational speed of the motor. The rotational speed of the motor may be inconsistent due to manufacturing tolerance and other factors. Motor speed inconsistency may cause some devices to emit higher dose and some lower dose depending on the speed of the motor.

To prevent such inconsistency the drive circuit that controls the motor can include an optical sensor that measures the rotational speed of the motor and inputs the value to a microprocessor where the measured speed is compared with the desired target speed. The circuit then increases or decreases the power delivered to the motor until it reaches the target speed. The speed of the motor converges to the desired target speed typically within less than 5 actuations and preferably less than two actuations.

The power delivered to the motor can be controlled by Pulse Width Modulation (PWM). PWM is a method of reducing the average power delivered to an electrical motor or other electrical load by effectively applying short and discrete DC pulses at high frequency, typically in the range of 5-15 KHz.

The voltage or current source can be supplied to the motor by means of a repeating series of “on” and “off” pulses. The on-time is the time during which the DC supply is applied to the motor, and the off-time is the period during which that supply is switched off. The density of the on time relative to the density of the off time controls the power delivered to the motor. The microprocessor increases or decrease the on time until the desired speed is reached.

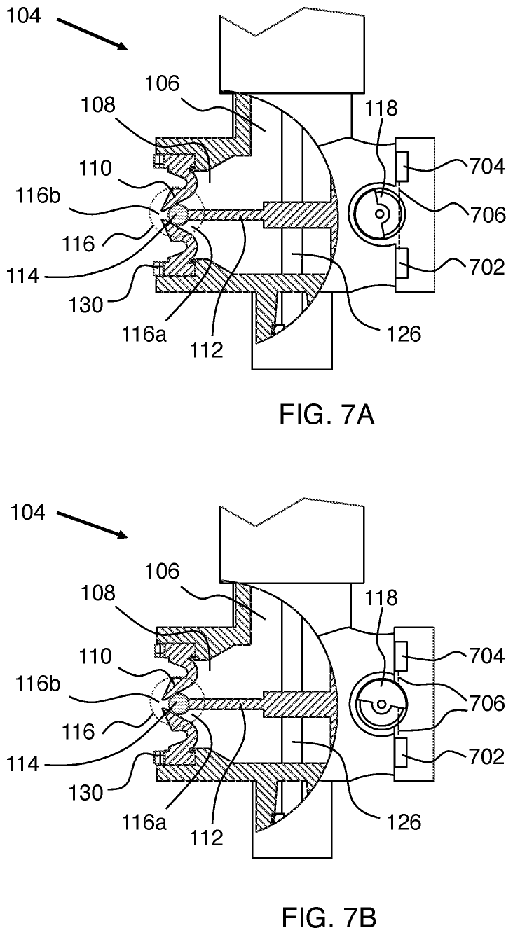

FIGS. 7A-B illustrate the optical sensors that measure the motor speed. The sensors include an LED 702 and a phototransistor 704 positioned coaxially and in predetermined distance from the rotational axis of the motor and its eccentric flywheel 118 such that the flywheel periodically blocks the light. In the first part of the revolution shown in FIG. 7A flywheel 118 is in a rotational position which allow passage of light beam 706 from the LED 702 to reach phototransistor 704 therefore causing the phototransistor to produce an electrical signal. At the second part of the revolution shown in FIG. 7B the light beam 706 from the LED is blocked and interrupted by the flywheel 118. The time between two subsequent interruption signals indicates the cycle time of the motor, the inverse of the cycle time is the number of revolutions per unit time (i.e., the frequency of the motor rotation (which is the same as the frequency of the above-described mechanical excitation)). In this way it is possible to measure the speed of the motor, compare it to the to the target value and make a correction as described earlier.

In this example, an LED is used as the light source and a phototransistor is used as the light detector, but practice of the invention does not depend critically on the choices of optical source and optical detector. Any sources and detectors can be employed.

What is claimed is:

1. A method for delivering fluid to an eye of a patient, the method comprising:
oscillating an actuator against a fluid containing chamber to generate fluid momentum therein;
measuring oscillation frequency of the actuator and providing such resulting measurements to a controller; and
using the controller to adjust an operating speed of the actuator to a level at which a desired volume of the fluid can be ejected through an aperture in the chamber to the eye of the patient.

2. The method of claim 1, wherein, in the oscillating step, the aperture and a pin member disposed within the chamber are hermetically sealed when the fluid is not being ejected.

3. The method of claim 2, further comprising:
biasing the aperture against the pin member via a compression spring.

4. The method of claim 1, wherein, in the using step, the aperture is a flexible aperture.

5. The method of claim 1, wherein the using step includes altering a power delivered to the actuator by Pulse Width Modulation.

6. The method of claim 1, wherein, in the measuring step, the oscillation frequency of the actuator is measured through a sensor.

7. The method of claim 6, wherein, in the measuring step, the sensor is an optical sensor.

8. The method of claim 1, further comprising:
aligning the aperture using a mirror disposed proximate to the aperture such that when the eye of the patient is in-focus in the mirror, as observed by the patient, ejection of the fluid through the aperture will be delivered accurately to the eye.

9. The method of claim 1, further comprising:
equalizing a pressure of the fluid as it is ejected through the aperture.

10. The method of claim 9, wherein the equalizing step includes a vent in fluid communication with the chamber and ambient air and introduces air into the chamber as the fluid is ejected.

11. The method of claim 10, further comprising:
filtering the air to ensure the air is sterile before introduction into the chamber.

12. An apparatus for delivering a fluid to an eye of a patient, the apparatus comprising:
a fluid containing chamber, the chamber having an aperture through which the fluid can be selectively dispensed;
an actuator engaging the chamber to generate fluid momentum in the fluid therein, the fluid momentum being sufficient to open the aperture and eject the fluid through the aperture; and
a controller configured to adjust an operating speed of the actuator to a level at which a desired volume of the fluid can be ejected through the aperture.

13. The apparatus of claim 12, wherein the actuator includes a vibration motor having an eccentric mechanical load relative to an axis of rotation of the vibration motor.

14. The apparatus of claim 12, wherein the aperture is tapered to be narrower at an outlet of the aperture than at an inlet of the aperture.

15. The apparatus of claim 12, further comprising a pin member engaging the aperture such that the aperture, as a result of the fluid momentum, is disengaged from the pin member.

16. The apparatus of claim 12, wherein the controller includes a sensor to measure the operating speed of the actuator.

17. The apparatus of claim 16, wherein the sensor is an optical sensor.

18. The apparatus of claim 12, further comprising a vent to equalize a pressure of the fluid as the fluid is ejected.

19. The apparatus of claim 18, wherein the vent includes a filter configured to ensure that, as ambient air is introduced, the ambient air that enters is sterile.

20. The apparatus of claim 12, further comprising a concave mirror configured to provide an in-focus image of the eye of the patient to the patient when the chamber is positioned properly to deliver the fluid to the eye of the patient.
